(19) United States
(12) Patent Application Publication
(10) Pub. No.: US 2009/0161817 A1
(43) Pub. Date: Jun. 25, 2009

(54) FAN-BEAM COHERENT-SCATTER COMPUTER TOMOGRAPH

(75) Inventor: Jens-Peter Schlomka, Hamburg (DE)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronic, N.V., Eindhoven (NL)

(21) Appl. No.: 11/568,118

(22) PCT Filed: Apr. 12, 2005
(86) PCT No.: PCT/IB05/51192

§ 371 Date: Oct. 20, 2006

(30) Foreign Application Priority Data
Apr. 21, 2004 (EP) 04101660.1

(51) Int. Cl. A61B 6/00

(52) U.S. Cl. 378/14

(57) ABSTRACT

However, this requires additional collimating means and this reduces the photon flux applied to the detectors. Due to this, longer measuring times may be required. Furthermore, the geometry is incompatible to known cone-beam CT-scanners. According to an exemplary embodiment of the present invention, a cone-beam CSCT scanner is provided using energy resolving detectors with a collimator arranged on the detectors, which allows spatially-resolved reconstruction of the scattering function. Advantageously, this may allow for an improved scanning speed in baggage inspection or medical applications.

The present invention relates to the field of coherent-scatter computer tomography (CSCT), where radiation such as x-rays is applied to an object of interest. In particular, the present invention relates to a computer tomography apparatus for examination of an object of interest, to a scatter radiation unit for a cone-beam computer tomography apparatus for examination of an object of interest and to a method of performing a cone-beam coherent scatter computer tomography scan.

U.S. Pat. No. 4,751,722 describes a device based on the principle of registration of an angled distribution of coherent scatter radiation within angles of 1° to 12° as related to the direction of the beam at X-ray energies around 100 keV. As set forth in U.S. Pat. No. 4,751,722, the main fraction of elastic scattered radiation is concentrated within angles of less than 12° and the scattered radiation has a characteristic angle dependency with well marked maxima, the positions of which are determined by the irradiated substance itself. As the distribution of the intensity of the coherent scatter radiation in small angles depends on the molecular structure of the substance, different substances having equal absorption capacity (which cannot be differentiated with conventional transillumination or CT) can be distinguished according to the intensity of the angled scattering of coherent radiation typical for each substance.

Due to the improved capabilities of such systems to distinguish different object materials, such systems find more and more application in medical or in industrial fields.

The dominant component of low-angle scatter is coherent scatter. Because coherent scatter exhibits interference effects which depend on the atomic arrangement of the scattering sample, coherent scatter computer tomography (CSCT) is in principle a sensitive technique for imaging spatial variations in the molecular structure of tissues across a 2D object section.

Harding et al “Energy-dispersive x-ray diffraction tomography” Phys. Med. Biol., 1990, Vol. 35, No. 1, 33-41 describes an energy dispersive x-ray diffraction tomography (EXDT) which is a tomographic imaging technique based on an energy analysis at fixed angle, of coherent x-ray scatter excited in an object by polychromatic radiation.

According to this method, a radiation beam is created by the use of suitable aperture systems, which has the form of a pencil and thus is also referred to as a pencil beam. Opposite to the pencil beam structure, one detector element suitable for an energy analysis is arranged for detecting the pencil beam altered by the object of interest.

Due to the use of the pencil beam in combination with only one or a few detector elements, only a limited number of photons emitted by the source of radiation and thus only a reduced amount of information can be measured. In case EXDT is applied to larger objects, such as, for example, to pieces of baggage, EXDT has to be used in a scanning mode, thus causing extremely long measurement times.

A coherent scatter set-up applying a fan-beam primary fan and a 2-dimensional detector in combination with CT was described in U.S. Pat. No. 6,470,067 B1 thus overcoming the long measurement time involved in EXDT scanning mode. The shortcoming of the angle-dispersive set-up in combination with a polychromatic source are blurred scatter functions, which are described, for example, in Schneider et al. “Coherent Scatter Computed Tomography applying a Fan-Beam Geometry” Pro. SPIE, 2001, Vol. 4320 754-763.

Still, there is a need for fast coherent scatter CTs.

It is an object of the present invention to provide for a fast coherent scatter computer tomography apparatus.

According to an exemplary embodiment of the present invention as set forth in claim 1, a computer tomography apparatus for examination of an object of interest is provided, wherein the computer tomography apparatus comprises a source of radiation, a scatter radiation detector for receiving a scatter radiation scattered by the object of interest and a first collimator. The scatter radiation detector is arranged opposite to the source of radiation with an offset with respect to a central plane, extending through the object of interest and the source of radiation. The scatter radiation has a plurality of regions. Each of the regions comprises at least one first detector element. The first detector elements are energy resolving detector elements. The first collimator is adapted such that radiation impinging on the at least one first detector element of a respective region of the plurality of regions is substantially restricted to radiation scattered from a predetermined section of the object of interest. The source of radiation is adapted to generate a cone-beam of radiation.

In other words, according to an aspect of this exemplary embodiment of the present invention, a CSCT apparatus is provided, applying a cone-beam. To allow for the spatial assignment of the received scattered radiation, the first collimator is provided together with the energy resolving scatter radiation, ensuring that only scatter radiation having a predetermined angle with respect to the source of radiation and with respect to the object of interest impinges onto the respective detector element of the scatter radiation detector. Thus, the energy resolving detector, i.e. the scatter radiation detector, measures the energy distribution of the scatter radiation scattered from the predetermined section of the object of interest. The predetermined section is determined by the arrangement of the collimator, i.e. of the focus of the collimator. From this, a coherent scatter function may be determined which has a spatial resolution.

Advantageously, due to the use of the cone-beam, the scan time required may be greatly reduced.

According to another exemplary embodiment of the present invention as set forth in claim 2, the first collimator comprises a second collimator and a third collimator. The second collimator is focused at the source of radiation, whereas the third collimator is focused at the section of the object of interest. By arranging the first and second collimators in layers above the scatter radiation detector, or one after the other with respect to the source of radiation, the radiation impinging on the respective detector element of the scatter radiation detector may be restricted to radiation scattered in a predetermined small section or region of the object of interest. In other words, by applying the second and third collimators, the first collimator may be realized such that each detector element of the scatter radiation detector associated with the first collimator has a predetermined “line of vision” of the object of interest.

According to another exemplary embodiment of the present invention as set forth in claim 3, the second and third collimators are realized by using lamellae, which are focused at the source of radiation for the second collimator and which are focused at the section of interest of the object of interest such that the “view” of the respective detector elements associated with the respective portion of the first collimator have a predetermined line of vision.

According to another exemplary embodiment of the present invention as set forth in claim 4, the second and third collimators are implemented by means of a slot collimator comprising holes which, for each respective region or for each respective detector element associated therewith are respectively focused at the source of radiation and the section of the object of interest. This may allow for a first collimator having a simple and robust arrangement.

According to another exemplary embodiment of the present invention as set forth in claim 5, a primary radiation detector is provided in the central plane for receiving a primary radiation attenuated by the object of interest. Advantageously, this may allow to collect scatter radiation data and attenuation data at the same time, i.e. during the same scan, and to use the attenuation data for compensating the scatter radiation data. Advantageously, this may allow for very accurate scanning results.

According to another exemplary embodiment of the present invention as set forth in claim 6, the energy resolving elements are direct converting semi-conductor cells and the primary radiation cells are scintillator cells.

According to another exemplary embodiment of the present invention as set forth in claim 7, the scatter radiation detector and the primary radiation detector are either integrated into one detector unit or are arranged as separate detector units, which also may be attached to the computer tomography apparatus independently.

According to another exemplary embodiment of the present invention as set forth in claim 8, a scatter radiation unit is provided which may be arranged in a cone-beam computer tomography apparatus for the examination of an object of interest. The scatter radiation unit comprises a scatter radiation detector and a first collimator. The scatter radiation detector is adapted for attachment to the cone-beam computer tomography apparatus such that the scatter radiation detector is arranged for receiving a scatter radiation scattered by the object of interest. The first collimator is adapted for arrangement with the scatter radiation detector. The scatter radiation detector is adapted for an arrangement opposite to the source of radiation of the cone-beam computer tomography apparatus with an offset with respect to a central plane extending through the object of interest and the source of radiation.

The scatter radiation detector has a plurality of regions, wherein each of the regions has at least one first detector element. The first detector elements are energy resolving detector elements. The first collimator is adapted such that radiation impinging on the at least one first detector element of a respective region of the plurality of regions is substantially restricted to a radiation scattered from a predetermined section of the object of interest. The source of radiation is adapted to generate a cone-beam of radiation.

Advantageously, this scatter radiation unit may be arranged in a known cone-beam CT scanner, such that a known cone-beam CT scanner, such as known from U.S. Pat. No. 6,269,141 B1 may advantageously be transferred to a cone-beam CSCT scanner. No primary radiation aperture systems are required.

This may allow for a very simple constitution and furthermore may allow to upgrade known cone-beam CT scanners to cone-beam CSCT scanners.

According to another exemplary embodiment of the present invention as set forth in claim 9, the first collimator comprises second and third collimators, allowing the line of vision of energy resolving cells of the scatter radiation detector to a predetermined section of the object of interest.

It should be noted that preferably each of the energy resolving detector elements of the scatter radiation detector has its own line or small volume of vision, which do not intersect within the region of interest. The width of the lines of vision determine the spatial resolution of the cone-beam CSCT apparatus.

According to another exemplary embodiment of the present invention as set forth in claim 10, the second and third collimators are realized by means of accordingly arranged lamellae. This may allow for a simple arrangement of the scatter radiation unit.

According to another exemplary embodiment of the present invention as set forth in claim 11, the second and third collimators may be realized by means of a slot collimator.

According to another exemplary embodiment of the present invention as set forth in claim 12, the scatter radiation unit is adapted for an arrangement with a primary radiation detector of the cone-beam radiation detector. For this, for example, the scatter radiation unit may also comprise the primary radiation detector, such that when, for example, a cone-beam CT is converted to a cone-beam CSCT, the whole detector unit is exchanged. However, the scatter radiation unit may also be provided without the primary radiation detector, such that, for upgrading the cone-beam CT to the cone-beam CSCT, only the scatter radiation unit is arranged accordingly in the cone-beam CT apparatus.

According to another exemplary embodiment of the present invention as set forth in claim 13, the energy resolving detector elements of the scatter radiation detector are direct converting semi-conductor cells.

According to another exemplary embodiment of the present invention as set forth in claim 14, a method of performing a cone-beam coherent scatter computer tomography scan with a computer tomography apparatus for examination of an object of interest is provided. According to this method, a source of radiation is provided. A scatter radiation detector is provided for receiving a scatter radiation scattered by the object of interest. Also, a first collimator is provided. The scatter radiation detector is arranged opposite to the source of radiation with an offset with respect to a central plane, extending through the object of interest and the source of radiation. The scatter radiation detector has a plurality of regions, each having at least one first detector element, preferably constituted as an energy resolving detector element. The first collimator is adapted such that radiation impinging on a region of the plurality of regions is substantially restricted to a radiation scattered from a predetermined section of the object of interest. In other words, the first collimator may be adapted such that each region of the energy resolving detector elements associated with a region always have a line of vision with respect to the object of interest, such that scatter radiation scattered only within such a section of the object of interest impinges onto these energy resolving detector elements.

According to this exemplary embodiment of this method, the source of radiation is energized so as to generate a cone-beam of radiation. Then, readouts from the scatter radiation detector are determined. The readouts from the scatter radiation detector are subjected to an absorption correction. Subsequently, a reconstruction of a coherent scatter function is performed on the basis of the corrected readouts.

Advantageously, a very fast method may be provided.

According to another exemplary embodiment of the present invention as set forth in claim 15, attenuation coefficients of the object of interest are determined by using readouts of a primary radiation detector arranged in the central plane. Then, parameters for the absorption correction of the readouts from the scatter radiation detector are determined on the basis of the attenuation coefficients. This may allow for a very accurate scanning result, for example for a good image quality of a reconstructed image.

According to another exemplary embodiment of the present invention as set forth in claim 16, the source of radiation is operated such that the primary radiation detector and the scatter radiation detector are subjected to the cone-beam radiation emitted from the source of radiation essentially at the same time.

Due to the gathering of attenuation data and scatter data at the same time, a fast scanning method may be provided.

It may be seen as the gist of an exemplary embodiment of the present invention that a cone-beam CSCT is provided. By using, for example, 2-dimensional collimators in combination with, for example, 2-dimensional energy resolving detectors, a reconstruction of the scattering function of an object of interest illuminated by the cone-beam may be possible. Advantageously, this may allow for a compatibility with cone-beam CT, i.e. additional slits, for example for a separate (fan-beam) primary beam, are no longer necessary. Instead, according to an exemplary embodiment of the present invention, cone-beam transmission CT and cone-beam CSCT are measured simultaneously. According to an aspect, cone-beam CSCT functionality may be added to a conventional cone-beam CT scanner by fitting the conventional cone-beam CT scanner with additional energy resolving detector units, also comprising the collimator according to the present invention.

These and other aspects of the present invention will become apparent from and elucidated with reference to the embodiments described hereinafter.

Exemplary embodiments of the present invention will be described in the following, with reference to the following drawings:

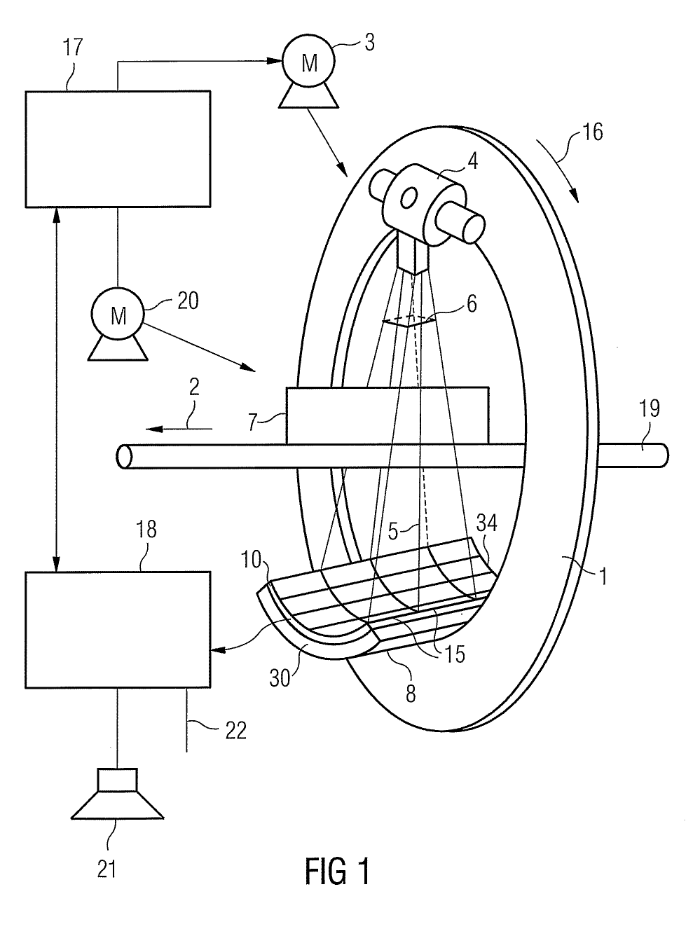

FIG. 1 shows a schematic representation of an exemplary embodiment of a cone-beam coherent scatter computer tomograph according to the present invention.

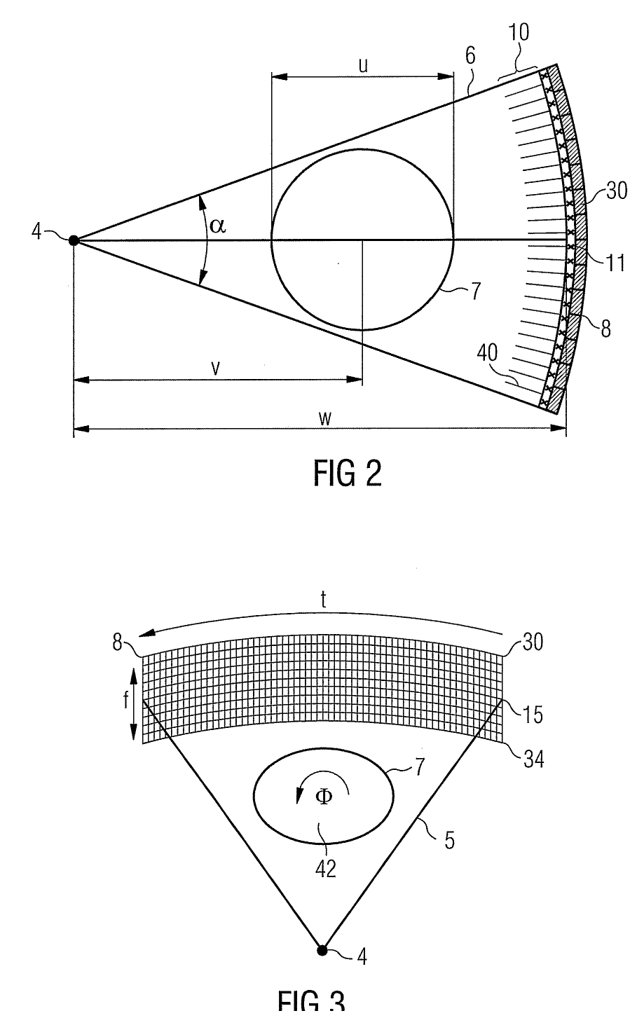

FIG. 2 shows a schematic representation of the geometry of the computer tomograph of FIG. 1 for the measurement of coherent scatter radiation.

FIG. 3 shows another schematic representation of the geometry of the computer tomograph of FIG. 1.

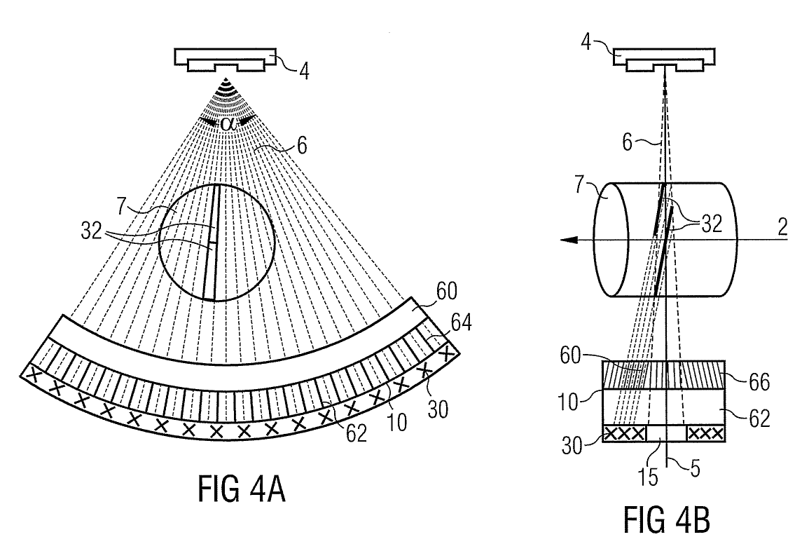

FIG. 4a shows a schematic representation of the source of radiation collimator and detector arrangement of the computer tomography of FIG. 1 in the center plane.

FIG. 4b shows a schematic representation of a side view onto the central plane for further explaining the arrangement of the source of radiation of the collimator and the detector according to an exemplary embodiment of the present invention.

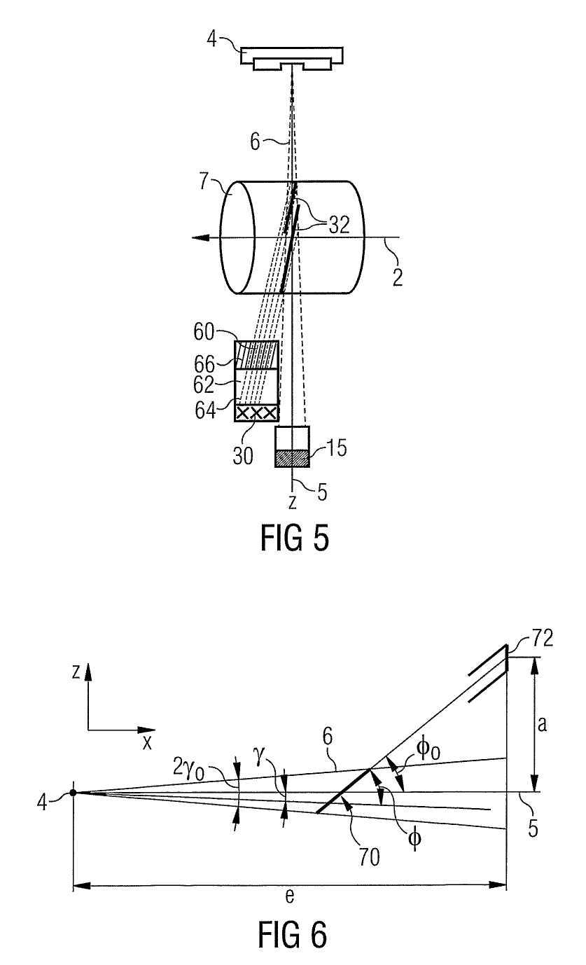

FIG. 5 shows another schematic representation of a side view of the central plane of an arrangement of the source of radiation, scatter radiation and primary radiation according to an exemplary embodiment of the present invention.

FIG. 6 shows another schematic representation of the measurement geometry according to an exemplary embodiment of the present invention as it may be applied in the computed tomograph according to the present invention.

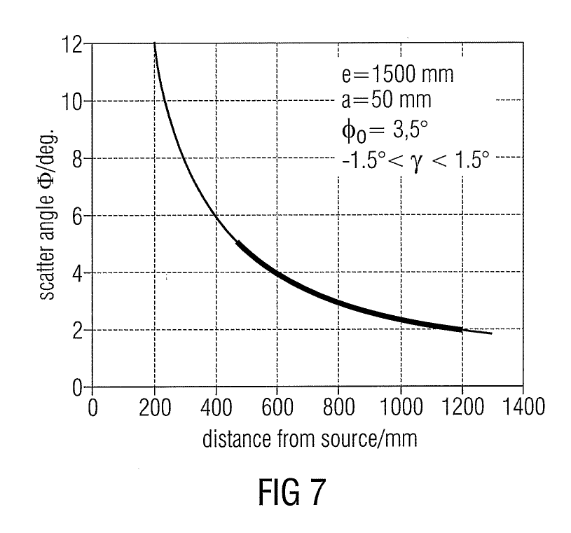

FIG. 7 shows a scatter angle with a distance from source diagram depicting an example of a calculation of a scatter angle independent of a distance of a point of interaction from the source of radiation.

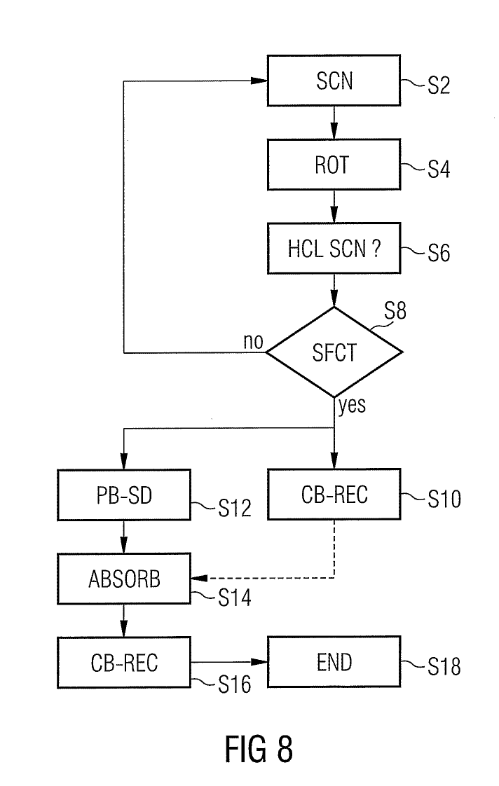

FIG. 8 shows a simplified flow-chart of a method of operating an exemplary embodiment of a computed tomograph according to the present invention.

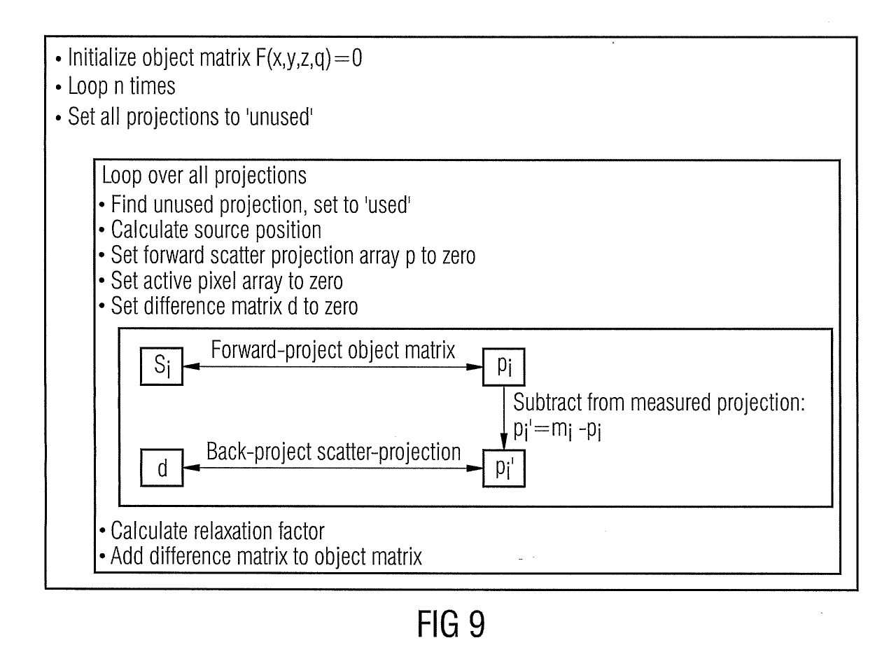

FIG. 9 shows a diagram of a reconstruction routine as it may be applied in step S16 of the method depicted in FIG. 8.

In the following description of FIGS. 1 to 9, the same reference numerals are used for the same or corresponding elements.

FIG. 1 shows an exemplary embodiment of a cone-beam coherent scatter computer tomography apparatus (cone-beam CSCT) according to the present invention. With reference to this exemplary embodiment, the present invention will be described for the application in baggage inspection to detect hazardous materials such as explosives in items of baggage. However, it has to be noted that the present invention is not limited to applications in the field of baggage inspection, but can also be used in other industrial or medical applications, such as for example in bone imaging or a discrimination of tissue types in medical applications. Also, it should be noted that the present invention is not limited to scanners having a rotating gantry. It may also be applied to scanners with stationary gantry.

The computer tomograph depicted in FIG. 1, as already indicated above, is a cone-beam coherent scatter computed tomograph (CSCT), which allows, in combination with energy-resolving detectors and with a tomographic reconstruction, a good spectral resolution with a polychromatic cone-beam. The computer tomograph depicted in FIG. 1 comprises a gantry 1, which is rotatable around a rotational axis 2. The gantry 1 is driven by means of a motor 3. Reference character 4 designates a source of radiation, such as an x-ray source, which is adapted to emit a cone-beam 6 of radiation.

The cone-beam 6 is directed such that it penetrates an item of baggage 7 arranged in the center of the gantry 1, i.e. in an examination region of the computer tomography and impinges onto collimator 10 which is arranged on detector 8. As may be taken from FIG. 1, the collimator 10 and the detector 8 are arranged on the gantry 1 opposite to the radiation source 4 such that a central plane 5 intersecting the source of radiation 4 and the item of baggage 7 intersects a row or line preferably in the center of the detector 8. The detector 8 depicted in FIG. 1 has a plurality of detector lines, each comprising a plurality of detector elements. Due to the arrangement of the collimator in the detector in FIG. 1, the surface of the detector 8 is covered by the collimator 10 such that the arrangement of detector elements of the detector is covered in FIG. 1.

The detector 8 comprises two types of radiation detector lines: a first type of detector lines 30 and 34, which are detector lines consisting of energy resolving detector cells. They are arranged such that they are outside the surface of the detector 8 which is subjected to a direct illumination by the cone beam 6. According to an aspect of the present invention, these first detector elements (lines 30 and 34) are energy-resolving detector elements. Preferably, the energy resolving detector elements are direct-converting semiconductor detector cells. Direct-converting semiconductor detector cells directly convert the radiation into electrical charges—without scintillation. Preferably, these direct-converting semiconductor detectors have an energy resolution better than 20% FWHM, i.e. ΔE/E<0.2, with ΔE being the Full-Width at Half Maximum (FWHM) of the energy resolution of the detector. The energy resolving detector elements may also be distributed in a non ordered fashion, i.e. not in lines.

Such detector cells of lines 30 and 34 may be cadmiumtelluride or CZT based detector cells, which are both outside of the central plane 5 of the cone-beam 6. In other words, all energy resolving lines 30 and 34 are arranged at the gantry 1 opposite to the x-ray source 4 with an offset from the central plane 5 in a direction parallel to the rotational axis 2. The detector lines 30 are arranged with a positive offset with respect to the direction of the rotational axis 2 depicted in FIG. 1, whereas the lines 34 are arranged with a negative offset from the central plane with respect to the direction of the rotational axis 2 depicted in FIG. 1. Also, as set forth above, the energy resolving detector elements are preferable arranged in regions of the detector 8 which are not subjected to direct illumination by the cone beam 6 such that they are adapted to measure the scatter radiation, i.e. the radiation scattered from the item of interest 7.

The detector lines 30 and 34 are arranged at the gantry 1 such that they are parallel to the central plane 5 with an offset in a positive or negative direction of the rotational axis 2 of the gantry 1, such that they receive or measure a scatter radiation scattered from the item of baggage 7 in the examination area of the computer tomograph. Thus, in the following, lines 30 and 34 will also be referred to as scatter radiation detector.

It has to be noted that instead of the provision of a plurality of energy resolving lines 30 and 34 on both sides of the central plane 5, it may also be efficient to provide only a reduced number of lines on only one side of the central plane 5.

Thus, if, in the following the term “scatter radiation detector” is used, it includes any detector with a 2-dimensional arrangement of energy resolving detector elements, which are arranged out of the central plane 5 of the cone-beam 6, such that they receive photons scattered from the item of baggage 7.

The second type of detector lines provided on the detector 8, are scintillator cells. In particular, lines 15 of scintillator cells are arranged on areas of the detector 8 which are subjected to direct illumination by the cone-beam 6. As indicated in FIG. 1 lines 15 may be arranged in a central area of the detector intersected by the central plane 5. Lines 15 may be parallel to the central plane 5. In other words, line 15 is arranged for measuring the attenuation of the radiation emitted by the source of radiation caused by the item of baggage 7 in the examination region.

As already indicated with respect to the energy resolving lines 30 and 34, where the provision of only a few energy resolving line 30 or 34 may be sufficient, the provision of only a few lines 15 measuring the attenuation caused by the item of baggage 7 of the primary beam of the cone-beam 6 in the central plane 5 may be sufficient. However, as in the case of the energy resolving lines 30 and 34, a provision of a plurality of detector lines 15, each comprising a plurality of scintillator cells, may further increase the measurement speed of the computer tomograph. In the following, the term “primary radiation detector” will be used to refer to a detector, including at least one scintillator cell or similar detector cells for measuring an attenuation of the primary radiation of the cone-beam 6.

Preferably, the detector cells of the detector 8 are arranged in lines and columns, wherein the columns are parallel to the rotational axis 2, whereas the lines are arranged in planes perpendicular to the rotational axis 2 and parallel to the central plane 5 of the cone-beam 6.

Furthermore, aperture systems (not shown in FIG. 1) may be provided to limit the dimensions of the cone-beam 5, such that no excess radiation is applied to the item of baggage 7, i.e. such that radiation not impinging onto the detector 8 may be cut away.

During a scan of the item of baggage 7, the radiation source 4 and the detector 8 are rotated along the gantry 1 in the direction indicated with arrow 16. For rotation of the gantry 1 with the source of radiation 4 and the detector 8, the motor 3 is connected to a motor control unit 17, which is connected to a calculation unit 18.

In FIG. 1, the item of baggage 7 is disposed on a conveyor belt 19. During the scan of the item of baggage 7, while the gantry 1 rotates around the item of baggage 7, the conveyor belt 19 may displace the item of baggage 7 along a direction parallel to the rotational axis 2 of the gantry 1. By this, the item of baggage 7 may be scanned along a helical scan path. However, the conveyor belt 19 may also be stopped during the scans to thereby measure single slices.

The detector 8 is connected to a calculation unit 18. The calculation unit 18 receives the detection results, i.e. the readouts from the detector elements of the detector 8 and determines a scanning result on the basis of the scanning results from the detector 8, i.e. from the energy resolving lines 30 and 34 and the line 15 for measuring the attenuation of the primary radiation of the cone-beam 6. In addition to that, the calculation unit 18 communicates with the motor control unit 17 in order to coordinate the movement of the gantry 1 with the motors 3 and 20 or with the conveyor belt 19.

The calculation unit 18 may be adapted for reconstructing images from readouts of the primary radiation detector, i.e. detector line 15 and the scatter radiation detector, i.e. lines 30 and 34. The images generated by the calculation unit 18 may be output to a display (not shown in FIG. 1) via an interface 22.

Furthermore, the calculation unit 18 may be adapted for the detection of explosives in the item of baggage 7 on the basis of the readouts of the lines 30 and 34 and 15. This may be made automatically by reconstructing scatter functions from the readouts of these detector lines and comparing them to tables including characteristic measurement values of explosives determined during preceding measurements. In case the calculation unit 18 determines that the measurement values read out from the detector 8 match with characteristic measurement values of an explosive, the calculation unit 18 may automatically output an alarm via a loudspeaker 21.

As indicated above, reference numeral 10 in FIG. 1 designates a collimator. The collimator is arranged above the detector elements of the detector 8. The collimator 10 is arranged such that each detector element detects only radiation from a section of the item of baggage 7 having the form of a ray. This ray is determined from a cross-section of the illuminated volume of the item of baggage 7 and by the section of the item of baggage 7 seen by the respective detector element. In other words, the collimator 10, which may be a 2-dimensional collimator, as depicted in FIG. 1, ensures that only scatter radiation having a predetermined angle may be detected by the detector. In other words, the collimator 10 may be adapted such that radiation impinging onto a detector element of one of the energy resolving detector elements is substantially restricted to radiation scattered from a predetermined section of the item of baggage 7. Due to the fact that it is known which detector element looks at which section of the item of baggage 7, the energy distribution measured by the respective energy resolving detector element may be assigned to a predetermined coordinate in the item of baggage 7. Thus, the energy resolving detector measures the energy distribution of the scatter radiation from the predetermined section of the item of baggage 7 at a predetermined line of vision. From this, a coherent scatter function having a spatial resolution may be determined by the calculation unit 18. This will be described in further detail with reference to FIGS. 2, 3 and 4.

FIG. 2 shows a simplified schematic representation of a geometry of the CSCT scanning system depicted in FIG. 1. As may be taken from FIG. 2, the x-ray source 4 emits the cone-beam 6 such that it includes the item of baggage 7 in this case having a diameter of u and covers the entire detector 8. The diameter of the object region may, for example, be 100 cm. In this case, an angle α of the cone-beam 6 may be 80°. In such an arrangement, a distance v from the x-ray source 4 to the center of the object region is approximately 80 cm and the distance of the detector 8, i.e. of the individual detector cells from the x-ray source 4 is approximately w=150 cm.

FIG. 2 shows a cross-sectional view of a slice where the cone-beam 6 is offset from the central plane 5, such that the slice depicted in FIG. 2 intersects energy resolving detector elements of one of lines 30. Reference numeral 10 designates the collimator, including first lamellae 40 and second lamellae 11.

As may be taken from FIG. 2, according to an aspect of the present invention, the detector cells or lines may be provided with lamellae 40 (or collimators) to avoid that the cells or lines measure unwanted radiation having a different scatter angle. The lamellae 40, which may also be referred to as collimators, may also have the form of blades, which can be focused towards the source of radiation 4. The spacing of the lamellae can be chosen independently from the spacing of the detector elements.

Furthermore, as indicated by reference numeral 11, a second row of lamellae may be provided between the first lamellae 40 and the detector 8. Preferably, these second lamellae are orientated such that they are focused at a predetermined section of the item of baggage 7.

Due to the different focus of the respective lamellae 11 and 40, it may be ensured that only radiation having a fixed predetermined angle impinge onto the detector and that each detector element only detects the scatter radiation from a predetermined oblong section of the item of baggage 7.

This will be described in further detail with reference to FIGS. 4a and 4b.

FIG. 3 shows another schematic representation of a detector geometry as used in the computer tomograph of FIG. 1. As already described with reference to FIG. 1, the detector 8 may comprise a plurality of energy resolving detector lines 30 and 34 and a plurality of lines 15 for measuring the attenuation of the primary cone-beam caused by the item of baggage 7. As may be taken from FIG. 3, preferably the detector 8 is arranged such that a central line of lines 15 is intersected and parallel to the central plane 5 of the cone-beam 6 and thereby measures the attenuation of the primary radiation. As indicated by arrow 42, the radiation source of x-ray source 4 and the detector 8 are rotated together around the item of baggage 7 to acquire projections from different angles. As depicted in FIG. 3, the detector 8 comprises a plurality of columns t.

Instead of a bent detector 8, as depicted in FIGS. 1, 2 and 3, it is also possible to use a flat detector array.

FIGS. 4a and 4b show top (FIG. 4a) and cross-sectional (FIG. 4b) views of the source of radiation, collimator and detector arrangement according to an exemplary embodiment of the present invention as it may be used in a computer tomography apparatus, as, for example, described with reference to FIG. 1.

As may be taken from FIGS. 4a and 4b, the energy resolving detector elements 30 are located behind a two-part collimator 10, comprising a first collimator portion 60 and a second lamellae portion 62. A part of the spatial resolution may be achieved by provision of focused lamellae 64 in the second collimator portion 62. The focused lamellae 64 are focused at the source of radiation 4. Furthermore, the focused lamellae 64 are arranged essentially perpendicular to the central plane 5. Due to this, only photons which have been scattered out of the central plane 5 may be detected by the energy resolving detector elements of detector lines 30 (or 34). Other photons, i.e. photons having another direction are absorbed by the focused lamellae 64 of the second collimator portion 62.

Thus, photons which are detected by a particular detector element of lines 30 and 34 may therefore only be scattered in a narrow restricted section of the probe. Such regions or sections are indicated by reference numeral 32 in FIGS. 4a and 4b.

A further part of the spatial resolution, namely the angular resolution, may be achieved by means of the first collimator portion 60, comprising further lamellae 66. These further lamellae 66, which are also focused, define a fixed line of vision of a respective energy resolving detector element onto the item of baggage 7. By this the origin of a photon detected in one particular detector element is restricted to a small portion of the object, essentially a line.

Due to the provision of the first and second collimator portions 60 and 62, i.e. due to the provision of focused lamellae 64 and 66, it may be achieved that only radiation of a fixed angle Φ₀ with respect to the central plane impinges onto the respective energy resolving detector elements. Furthermore, by the arrangement of lamellae 64 and 66, one after another, it may be achieved that each detector element (or each group of detector elements) may only detect scatter radiation from an oblong section 32 of the item of baggage 7. The position, orientation and size of the oblong section 32 may be set by an according arrangement of lamellae 64 and 66.

Instead of the provision of lamellae as first and second collimator portions 66 and 64, the collimator may also be realized by means of a so-called slot-collimator, consisting of, for example, a solid object having a strong absorbance with respect to x-rays, being provided with focused holes. Behind these focused holes, respective energy resolving detector elements may be provided.

Instead of a solid object, such a slot collimator may also be realized by a plurality of stapled aperture plates. Furthermore, the collimator (also collimator portions 60 and 62) may be realized by crossing lamellae.

In general, according to an exemplary embodiment of the present invention, the collimator shall be realized such that each detector pixel has only one “line of vision”. In the case of the slot collimator being provided with holes, for example along the lines of the detector 8, the lines may be focused onto the source of radiation 4, whereas, along the columns of the detector 8, all holes may be parallel to each other, each defining a constant angle Φ₀ with respect to the central plane.

Preferably, only one detector element is provided for each hole.

As may be taken from FIG. 4b, there may also be provided a conventional CT detector 15, which may, for example, be provided with a one-dimensional or two-dimensional anti-scatter grid, which may also be focused onto the source of radiation 4. The energy resolving detector elements may be provided on both sides of this primary radiation detector, which may allow for a higher yield of photons, but may also be provided on one side of the primary radiation detector, which may allow for reduced costs.

FIG. 5 shows another schematic representation of another exemplary embodiment of a cone-beam CSCT according to an exemplary embodiment of the present invention. In contrast to the embodiment depicted in FIGS. 4a and 4b, as may be taken from FIG. 5, the primary radiation detector (detector line 15) and the scatter radiation detector (detector lines 30) including a collimator with a first collimator portion 60 and a second collimator portion 62, each being provided with lamellae 64 and 66 are provided separately. According to an aspect of this exemplary embodiment, for example, the scatter radiation detector may be added to a known cone-beam CT for upgrading the cone-beam CT to a cone-beam CSCT, as depicted in FIG. 5. For this, a two-dimensional energy resolving detector unit, including a collimator according to an exemplary embodiment of the present invention, for example including a first collimator portion 16 and a second collimator portion 62, with lamellae 62 and 66 may be provided separately, which may be attached to an existent CT scanner. For this, the scatter radiation detector may be adapted for attachment to such cone-beam CT. The position of the scatter detector can be either closer to the source of radiation as shown in the exemplary embodiment, but can also be at a larger distance from the source.

FIG. 6 shows another schematic representation of the geometry of the source of radiation and the energy resolving detector element, according to an exemplary embodiment of the present invention for further explaining a method for calculating a scatter angle in dependence of the location of the scatter event according to an exemplary embodiment of the present invention. Reference numeral 4 designates the source of radiation, reference numeral 70 designates a scatter event and reference numeral 72 designates an energy resolving detector element, for example of detector line 70. Schematically, lamellae are depicted in front of the energy resolving detector element 72.

A distance between the source of radiation 4 and a central plane of the detector 8 is designated by e. A distance of the energy resolving detector element 72 from the central plane 5 is indicated by a. With respect to the central plane 5, the detector's vision line is at an angle Φ₀ with respect to the central slice. The cone angle is 2γ₀. In FIG. 6, a point of interaction 70 within the volume of the item of baggage 7 is contemplated, which line between the source of radiation 4 and the central plane 5 defines the angle γ. γ has values between −□₀ and □₀. The respective scatter angle is referred to as (d. The dependence of this scatter angle of the spatial coordinate x (distance from the source of radiation to the scatter event 70) may be calculated.

The scatter angle is dependent on the location of the scatter event, since the primary radiation is divergent. This may be taken from FIG. 6. However, since the dependence of the scatter angle from the location is known from the depicted geometry, this may be taken into account during reconstruction. Thus, according to an exemplary embodiment of the present invention, by using the following equation

$$q = \frac{E}{hc} sin ( Φ / 2 ) \qquad Equation 1$$

the scatter angle Φ existing at the respective location is used for the calculation of the wave vector transfer q with E being the energy of the detected radiation and h being Planck's constant and c the speed of light.

The dependence of Φ with respect to the distance between source position and point of interaction x can be calculated as follows:

FIG. 6 shows that

Φ=Φ₀−γ Equation 2.

Now, the variable 70 is introduced, relating to the cone-angle. Thus

y₀=a−tan(Φ₀)e Equation 3.

With a, e and Φ₀ as described in FIG. 6.

Hence, γ(x) may be calculated as follows:

$$γ ( x ) = {tan}^{- 1} ( tan ( {Φ}_{0} ) + \frac{{y}_{0}}{x} ) \qquad Equation 4$$

From this, q(x) may be calculated from a predetermined geometry (e, Φ₀). In other words, from a given geometry (e, Φ₀), the dependence between the location of the interaction (distance from the source x) and the corresponding wave vector transfer may be determined for each respective detector element having a distance a from the central plane 5 by using Equations 1-4.

In the reconstruction algorithm this dependence may be used for the forward projection as well as for the back projection.

FIG. 7 shows calculation examples where parameters were used which are indicated in FIG. 6.

In detail, FIG. 7 shows a scatter angle with the distance from source diagram depicting an example of a calculation of the scatter angle in dependence on the distance of the point of interaction from the source. The thick line indicates the region that is covered by a cone beam of 2γ₀=3°, i.e. −1.5°<γ<+1.5°.

In the following, with reference to FIGS. 8 and 9, a method of operating the cone-beam CSCT apparatus according to the present invention will be described.

FIG. 8 shows a simplified flow-chart of an exemplary embodiment of a method of operating the cone-beam CSCT apparatus according to the present invention.

In step S2, the source of radiation 4 is activated, i.e. energized such that a cone-beam of radiation 16 is emitted, which penetrates through the item of baggage 7 and impinges on the detector 8. This scan may be performed at a particular rotation angle of the source detector arrangement on the gantry 1 around the rotation axis 2. In particular, cone-beam transmission CT data is measured by means of the primary radiation detector, i.e. line 15. At the same time, the two-dimensional energy resolving detector (i.e. the scanner radiation detector including lines 30 and 34) detects the scatter radiation. After the acquisition of these measurements for such projection, i.e. rotation angle, the source detector arrangement is rotated by a predetermined angle in step S4.

In case it is determined in step S6 that the cone-beam CSCT scanner operates according to a helical scan mode, the calculation unit 18 activates the conveyor belt 19 such that the item of baggage 7 is translated along the rotation axis 2 for a predetermined distance.

It is to be noted that referring to the source detector arrangement in the explanation of this exemplary embodiment of the method of the present invention, this includes that the collimator 10 is rotated and moved in accordance with the detector 8.

Then, as indicated by SFCT in step S8, it is determined whether sufficient projections have to be measured. In case it is determined in step S8 that further projections need to be acquired, the method continues to step S2, where, as indicated by SCN the source of radiation 4 is energized and scatter radiation data and primary radiation data is gathered by means of the detector 8. Then, in subsequent step S4, as indicated by ROT, the source detector arrangement is rotated by a predetermined rotational increment. Then, in subsequent step S6, as already indicated above, and as indicated by HCL SCN?, it is determined whether a helical scan is performed and, if a helical scan mode is set, a translation of the object of interest, i.e. of the item of baggage 7 is performed along the rotational axis 2. Then, the method continues to step S8.

If it is determined in step S8 that sufficient projections have been determined, the method continues to step S12 and step S10.

As indicated by CB-REC in step S10, the readouts of the primary radiation detector, i.e. the attenuation data, is subjected to a known cone-beam CT reconstruction, as known, for example, from U.S. Pat. No. 6,269,141 B1 (“Computer tomography apparatus with a conical radiation beam and a helical scanning trajectory”) and references therein, which is hereby incorporated by reference. This cone-beam CT reconstruction allows to determine an image of the attenuation coefficients, i.e. a CT image, which is then input to step S14.

In step S12, as indicated by PB-SD, a correction of the scatter radiation data, i.e. the readouts of the scatter radiation detector, is performed. This may also be referred to as primary beam correction of the scatter radiation data. Essentially the detected scatter spectra are normalized to the primary spectrum, thus eliminating the energy-dependent intensity variation of the primary bremsstrahlung-spectrum, particularly due to characteristic emission.

Then, in the subsequent step S14, the scatter radiation data from the scatter radiation detector is subjected to an attenuation correction on the basis of the CT image determined in step S10. This is indicated by ABSORB in step S14. Here, the spectral dependency of the attenuation is corrected for. Then, in the subsequent step S16, as indicated by CB-REC, the absorption corrected scatter radiation data is subjected to a reconstruction routine, which performs a reconstruction of the coherent scatter function for each illuminated object voxel. For this, routines or methods may be used, which are based on ART (algebraic reconstruction technique), as for example, known for CT applications from the book by G. T. Herman G T “Image Reconstruction from Projections”, Academic Press, New York, 1980, which is hereby incorporated by reference. An exemplary embodiment of such a reconstruction routine will be described in further detail with reference to FIG. 9.

During this reconstruction routine, the dependency of the scatter angle from the location of the scatter event is taken into account.

However, instead of a routine based on art, also a filtered back projection may be performed. Such filtered back projection is, for example, known from the same book by G. T. Hermann.

FIG. 9 shows a diagram of a reconstruction routine as it may be applied in step S16 of the method depicted in FIG. 8. As may be taken from FIG. 96 firstly the object matrix F(x, y, z, q)=0 is initialized. Then, for n-times, the following loop is performed.

All projections are set to “unused”. Then, a further loop is performed over all projections. During this second loop, the following operations are performed. Firstly, an unused projections is searched for. Once such a projection is found it is set to “used”. Then, the source position of this projection is calculated. Then, a forward scatter projection array p is set to 0. Then, an active pixel array is set to 0. Also, a difference matrix d is set to 0.

Then, a forward projection of the object matrix is performed. This means that a scattering is simulated on the detector. For this, a primary spectrum is assumed, comprising a plurality of energy intervals. For each energy interval of this primary spectrum and for each location within the object of interest, a corresponding q value is calculated, which reaches the detector. This is calculated by using equations 1 to 4 as indicated above. Then, an intensity distribution on the detector is calculated by using the coherent scatter function F²(x,y,z,q) of the object matrix.

As may be taken from FIG. 9, subsequently, the intensity distribution on the detector is subtracted from the actually measured data, as indicated by pᵢ′=m₁−pᵢ and this difference pᵢ′ is subjected to a back projection. The back project scatter projection is the reversed calculation of the forward projection. The values determined by each detector element and each detected energy are evenly distributed along a line, from which they may originate. By doing this, the wave vector transfer is determined in accordance with equations 1 to 4 for each energy and for each location. This determines along which line in (x,y,z,q)-space the backprojection is performed.

Then, subsequently, a relaxation factor may be calculated and the difference matrix formed therefrom may be added to the object matrix.

Thus, according to the present invention, a cone-beam CSCT apparatus and method may be provided. As indicated above, for example, a 2-dimensional collimator may be used in combination with a 2-dimensional energy resolving detector for reconstructing the scattering function from an object illuminated by a cone-beam. As already indicated above, the scatter radiation detector according to the present invention is compatible to a cone-beam CT and may be integrated into a known cone-beam CT apparatus. Advantageously, if the scatter radiation detector is arranged in a cone-beam CT for upgrading the cone-beam CT to a cone-beam CSCT, additional slits for the primary beam are no longer necessary. Instead, cone-beam transmission CT and cone-beam CSCT may be measured simultaneously. Circular and helical trajectories are feasible. Overall, a speed-up of the scanning process may be achieved.

1. A computer tomography apparatus for examination of an object of interest, the computer tomography apparatus comprising:
a source of radiation; a scatter radiation detector for receiving a scatter radiation scattered by the object of interest; and a first collimator; wherein the scatter radiation detector is arranged opposite to the source of radiation with an offset with respect to a central plane; wherein the central plane extends trough the object of interest and the source of radiation; wherein the scatter radiation detector has a plurality of regions;
wherein each of the regions has at least one first detector element; wherein the first detector elements are energy resolving detector elements; wherein the first collimator is adapted such that radiation impinging on the at least one first detector element of a respective region of the plurality of regions is substantially restricted to radiation scattered from a predetermined section of the object of interest; and wherein the source of radiation is adapted to generate a cone beam of radiation.

2. The computer tomography apparatus of claim 1, wherein the first collimator comprises:
a second collimator; and a third collimator; wherein the second collimator is focused at the source of radiation; wherein the third collimator is focused at the section of the object of interest; and wherein the second and third collimators are arranged one after another with respect to the source of radiation.

3. The computer tomography apparatus of claim 2, wherein the second collimator has first lamellae which are focused at the source of radiation and are arranged substantially perpendicular to the central plane such that radiation impinging on the at least one first detector element in the region of the plurality of regions associated with the second collimator is restricted to radiation having a first predetermined angle with respect to the source of radiation; and wherein the third collimator has second lamellae which are focused at the section of the object of interest such that radiation impinging on the at least one first detector element in the region of the plurality of regions associated with the third collimator is restricted to radiation having a second predetermined angle with respect to the section of the object of interest.

4. The computer tomography apparatus of claim 2, wherein the second and third collimators are together implemented by means of a slot collimator comprising holes which, for each respective region, are respectively focused at the source of radiation and the section of the object of interest.

5. The computer tomography apparatus of claim 1, further comprising:
a primary radiation detector; wherein the primary radiation detector is arranged opposite to the source of radiation in the central plane for receiving a primary radiation attenuated by the object of interest.

6. The computer tomography apparatus of claim 5, wherein the energy resolving detector elements are direct-converting semiconductor cells; and wherein the primary radiation detector comprises scintillator cells.

7. The computer tomography apparatus of claim 5, wherein the scatter radiation detector and the primary radiation detector are one of integrated into one detector unit and separated as separate detector units.

8. A scatter radiation unit for a cone beam computer tomography apparatus for examination of an object of interest, the cone beam computer tomography apparatus including a source of radiation, the scatter radiation unit comprising: a scatter radiation detector; and a first collimator; wherein the scatter radiation detector is adapted for attachment to the cone beam computer tomography apparatus such that the scatter radiation detector is arranged for receiving a scatter radiation scattered by the object of interest; wherein the first collimator is adapted for arrangement with the scatter radiation detector; wherein the scatter radiation detector is adapted for an arrangement opposite to the source of radiation with an offset with respect to a central plane; wherein the central plane extends through the object of interest and the source of radiation; wherein the scatter radiation detector has a plurality of regions; wherein each of the regions has at least one first detector element; wherein the first detector elements are energy resolving detector elements; and wherein the first collimator is adapted such that radiation impinging on the at least one first detector element of a respective region of the plurality of regions is substantially restricted to radiation scattered from a predetermined section of the object of interest wherein the source of radiation is adapted to generate a cone beam of radiation.

9. The scatter radiation unit of claim 8, wherein the first collimator comprises: a second collimator; and a third collimator; wherein the second collimator is adapted such that it is focused at the source of radiation when the second collimator is arranged in the cone beam computer tomography apparatus; wherein the third collimator is adapted such that it is focused at the section of the object of interest when the third collimator is arranged in the cone beam computer tomography apparatus; and wherein the second and third collimators are arrangeable one after another with respect to the source of radiation.

10. The scatter radiation unit of claim 9, wherein the second collimator has first lamellae which are focused at the source of radiation and are arranged substantially perpendicular to the central plane such that radiation impinging on the at least one first detector element in the region of the plurality of regions associated with the second collimator is restricted to radiation having a first predetermined angle with respect to the source of radiation; and wherein the third collimator has second lamellae which are focused at the section of the object of interest such that radiation impinging on the at least one first detector element in the region of the plurality of regions associated with the third collimator is restricted to radiation having a second predetermined angle with respect to the section of the object of interest.

11. The scatter radiation unit of claim 9, wherein the second and third collimators are together implemented by means of a slot collimator comprising holes which, for each respective region, are respectively focused at the source of radiation and the section of the object of interest.

12. The scatter radiation unit of claim 8, wherein the scatter radiation unit is adapted for an arrangement with a primary radiation detector of the cone beam radiation detector; wherein the primary radiation detector of the cone beam radiation detector is arranged opposite to the source of radiation in the central plane for receiving a primary radiation attenuated by the object of interest.

13. The scatter radiation unit of claim 8, wherein the energy resolving detector elements are direct-converting semiconductor cells.

14. A method of performing a cone beam coherent scatter computer tomography scan with a computer tomography apparatus for examination of an object of interest, the method comprising the steps of:
providing a source of radiation; providing a scatter radiation detector for receiving a scatter radiation scattered by the object of interest; providing a first collimator; wherein the scatter radiation detector is arranged opposite to the source of radiation with an offset with respect to a central plane; wherein the central plane extends trough the object of interest and the source of radiation; wherein the scatter radiation detector has a plurality regions; wherein each of the regions has at least one first detector element;
wherein the first detector elements are energy resolving detector elements; wherein the first collimator is adapted such that radiation impinging on a region of the plurality of regions is substantially restricted to radiation scattered from a predetermined section of the object of interest; energizing the source of radiation to generate a cone beam of radiation; determining readouts from the scatter radiation detector; performing an absorption correction of the readouts from the scatter radiation detector; and performing a reconstruction of a coherent scatter function on the basis of the corrected readouts.

15. The method of claim 14, further comprising the steps of:
determining attenuation coefficients of the object of interest by using readouts of a primary radiation detector arranged in the central plane; determining parameters for the absorption correction of the readouts from the scatter radiation detector on the basis of the attenuation coefficients.

16. The method of claim 16, wherein the source of radiation is operated such that the primary radiation detector and the scatter radiation detector are subjected to the cone beam radiation emitted from the source of radiation essentially at the same time.
